(19) United States
(12) Patent Application Publication
(10) Pub. No.: US 2026/0015377 A1
(43) Pub. Date: Jan. 15, 2026

(54) CRYSTALLINE FORM I OF REDUCED NICOTINAMIDE MONONUCLEOTIDE DISODIUM MONOHYDRATE AND PREPARATION METHOD THEREFOR

(71) Applicant: Greenanew (Shanghai) Biotech Co., Ltd., SHanghai (CN)

(72) Inventors: Yantao QI, Shanghai (CN); Changgang Tu, Shanghai (CN); Chunyi Liu, Shanghai (CN); Xiaoming Zhu, Shanghai (CN); Tao Li, Shanghai (CN); Bo Wang, Shanghai (CN)

(73) Assignee: Greenanew (Shanghai) Biotech Co., Ltd., Shanghai (CN)

(21) Appl. No.: 19/095,548

(22) Filed: Mar. 31, 2025

(30) Foreign Application Priority Data
Jul. 9, 2024 (CN) 202410912487.4

(51) Int. Cl. C07H 19/048; A61K 8/60; A61K 31/706; A61Q 19/08

(52) U.S. Cl. CPC C07H 19/048; A61K 8/606; A61K 31/706; A61Q 19/08

(57) ABSTRACT

The invention relates to a crystalline form I of the reduced nicotinamide mononucleotide disodium monohydrate (NMNH) as shown in formula I, and its preparation method. The crystalline form I exhibits X-ray powder diffraction peaks at 2θ angles of 10.502°±0.2°, 12.721°±0.2°, 20.036°±0.2°, and 21.633°±0.2° using CuKα radiation. This crystalline form I has advantages such as good stability, low hygroscopicity, ease of storage, and a simple preparation method, making it suitable for large-scale production.

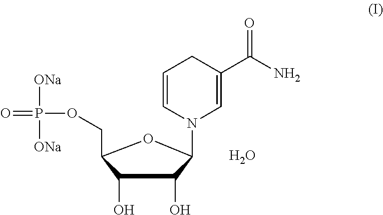

### TECHNICAL FIELD

The invention belongs to the field of pharmaceutical chemistry and specifically relates to a crystalline form and its preparation method of the reduced nicotinamide mononucleotide disodium monohydrate (NMNH).

### BACKGROUND ART

Nicotinamide adenine dinucleotide (NAD+) is an important coenzyme in cells and a core substance for anti-aging, capable of catalyzing over 500 enzymatic reactions in the human body. Increasing evidence shows that enhancing NAD+ levels can significantly improve the functions of multiple organs, including the liver, kidneys, heart, and skeletal muscles. The biosynthesis of NAD+ is achieved through nicotinamide (NAM), nicotinamide riboside (NR), and nicotinamide mononucleotide (NMN), with NMN being considered the most suitable NAD+ precursor at present. NMN is currently a hot-selling product in the global market, highly favored by consumers.

Reduced nicotinamide mononucleotide (NMNH) is the reduced form of NMN and serves as a new precursor for supplementing NAD+. Studies (The FASEB Journal, 2021, 35(4), e21456) have shown that NMNH is more effective than NMN or NR in increasing intracellular NAD+ levels. It is metabolized through a different pathway that does not depend on nicotinamide riboside kinase (NRK) and nicotinamide phosphoribosyltransferase (NAMPT), reducing damage to renal proximal tubular epithelial cells during hypoxia/reoxygenation injury and accelerating the repair process. As a new NAD+ precursor, NMNH has the potential to enhance NAD+ levels both in vivo and in vitro and may help improve diseases related to NAD+ metabolism.

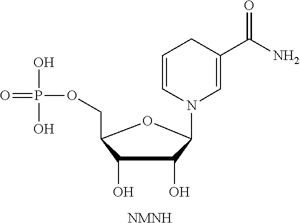

Currently, the preparation process of NMNH is difficult to industrialize. Products on the market are mostly amorphous solids obtained by freeze-drying aqueous solutions, which lack purification effects. Moreover, these freeze-dried solids are prone to moisture absorption, have poor flowability, and are easily degraded.

Patent CN115368423A reports several crystalline forms (crystalline forms A, B, and C) of the sodium salt of NMNH and their preparation methods and uses. However, the water content in these crystalline forms is not fixed, with significant fluctuations, which may lead to the presence of a large amount of adsorbed water and inevitably affect the storage stability of the products.

Therefore, there is an urgent need to develop a new crystalline form of NMNH with better stability for convenient long-term storage and transportation.

### CITATION LIST

Literature 1: The FASEB Journal, 2021, 35(4), e21456.

Literature 2: CN115368423A

### SUMMARY OF THE INVENTION

The inventors have conducted research on the salt forms and crystalline forms (including single and polymorphs) of NMNH. After extensive experimental exploration, a new crystalline form of NMNH with lower hygroscopicity and better thermal stability compared to the crystalline forms reported in CN115368423A has been developed.

Specifically, the invention provides the following technical solutions:

The invention provides a crystalline form I of the reduced nicotinamide mononucleotide disodium monohydrate as shown in formula I, which exhibits X-ray powder diffraction peaks at 2θ angles of 10.502°±0.2°, 12.721°±0.2°, 20.036°±0.2°, and 21.633°±0.2° using CuKα radiation. Preferably, it exhibits peaks at 10.502°±0.1°, 12.721°±0.1°, 20.036°±0.1°, and 21.633°±0.1°. More preferably, it exhibits peaks at approximately 10.502°, 12.721°, 20.036°, and 21.633°.

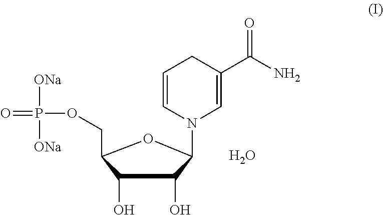

Furthermore, the crystalline form I also exhibits diffraction peaks at 2θ values of 12.134°±0.2°, 15.389°±0.2°, 20.461°±0.2°, and 25.639°±0.2°. It is preferred that these peaks are at 12.134°±0.1°, 15.389°±0.1°, 20.461° +0.1°, and 25.639°±0.1°. More preferably, the peaks are at approximately 12.134°, 15.389°, 20.461°, and 25.639°.

The crystalline form I further exhibits diffraction peaks at 2θ values of 15.994°±0.2°, 17.63°±0.2°, 18.096°±0.2°, 20.243°±0.2°, 23.29°±0.2°, and 31.389°±0.2°. Preferably, these peaks are at 15.994°±0.1°, 17.63°±0.1°, 18.096°±0.1°, 20.243°±0.1°, 23.29°±0.1°, and 31.389°±0.1°. More preferably, the peaks are at approximately 15.994°, 17.63°, 18.096°, 20.243°, 23.29°, and 31.389°.

An important indicator characterizing the crystalline form I is the X-ray powder diffraction pattern (XRPD), which is summarized in Table 1.

TABLE 1
XRPD of NMNH Disodium Monohydrate Crystalline Form I

| Peak No. | Position [°2θ] | d-spacing [Å] | Height [cts] | I/Imax [%] |
|---|---|---|---|---|
| 1 | 5.067 | 17.4257 | 37 | 4 |
| 2 | 5.365 | 16.4595 | 85 | 9.1 |
| 3 | 10.502 | 8.4168 | 228 | 24.4 |
| 4 | 11.648 | 7.5911 | 33 | 3.5 |
| 5 | 12.134 | 7.288 | 355 | 38 |
| 6 | 12.721 | 6.953 | 935 | 100 |
| 7 | 13.829 | 6.3981 | 41 | 4.4 |
| 8 | 14.582 | 6.0695 | 187 | 20 |
| 9 | 15.389 | 5.753 | 295 | 31.6 |
| 10 | 15.994 | 5.5366 | 242 | 25.9 |
| 11 | 16.605 | 5.3342 | 167 | 17.9 |
| 12 | 17.311 | 5.1184 | 38 | 4.1 |
| 13 | 17.63 | 5.0264 | 421 | 45 |
| 14 | 18.096 | 4.8981 | 357 | 38.2 |
| 15 | 19.555 | 4.5358 | 150 | 16 |
| 16 | 20.036 | 4.4279 | 438 | 46.8 |
| 17 | 20.243 | 4.3832 | 409 | 43.7 |
| 18 | 20.461 | 4.3369 | 414 | 44.3 |
| 19 | 21.026 | 4.2217 | 378 | 40.4 |
| 20 | 21.226 | 4.1823 | 219 | 23.4 |
| 21 | 21.633 | 4.1045 | 809 | 86.5 |
| 22 | 22.648 | 3.9229 | 149 | 15.9 |
| 23 | 23.29 | 3.8161 | 197 | 21.1 |
| 24 | 24.261 | 3.6656 | 103 | 11 |
| 25 | 24.827 | 3.5833 | 160 | 17.1 |
| 26 | 25.227 | 3.5274 | 73 | 7.8 |
| 27 | 25.639 | 3.4716 | 229 | 24.5 |
| 28 | 26.258 | 3.3912 | 61 | 6.5 |
| 29 | 26.583 | 3.3504 | 42 | 4.5 |
| 30 | 27.271 | 3.2674 | 110 | 11.8 |
| 31 | 27.815 | 3.2047 | 112 | 12 |
| 32 | 28.445 | 3.1352 | 93 | 9.9 |
| 33 | 28.732 | 3.1045 | 81 | 8.7 |
| 34 | 29.352 | 3.0403 | 66 | 7.1 |
| 35 | 29.574 | 3.018 | 60 | 6.4 |
| 36 | 30.93 | 2.8887 | 163 | 17.4 |
| 37 | 31.389 | 2.8475 | 37 | 4 |
| 38 | 31.821 | 2.8099 | 199 | 21.3 |
| 39 | 32.807 | 2.7276 | 66 | 7.1 |
| 40 | 33.534 | 2.6701 | 105 | 11.2 |
| 41 | 34.283 | 2.6135 | 86 | 9.2 |
| 42 | 36.791 | 2.4409 | 58 | 6.2 |
| 43 | 37.471 | 2.3981 | 40 | 4.3 |
| 44 | 38.145 | 2.3573 | 34 | 3.6 |

The term “relative intensity” refers to the ratio of the intensity of other peaks to the intensity of the highest peak when the intensity of the highest peak in the X-ray powder diffraction pattern is set to 100%.

In a preferred embodiment, the XRPD pattern of crystalline form I of the invention is substantially the same as that shown in FIG. 1.

Further, in DSC analysis, the crystalline form exhibits an endothermic peak in the range of 215.68˜251.58° C.; preferably, the maximum absorption peak is around 238.06° C.; more preferably, the DSC thermogram of the crystalline form is substantially as shown in FIG. 2.

Further, in TGA analysis, the weight loss is approximately 5.198% in the range of 50˜160° C. and approximately 43.877% in the range of 160˜500° C. Preferably, the TGA thermogram is substantially as shown in FIG. 3.

The invention also provides a method for preparing the crystalline form I, which is not limited to the steps described above, and various modifications or adjustments made by those skilled in the art within the scope of the claims should be included in the protection scope of the invention.

The second aspect of the invention provides a method for preparing the above-mentioned crystalline form I, which includes the following steps:

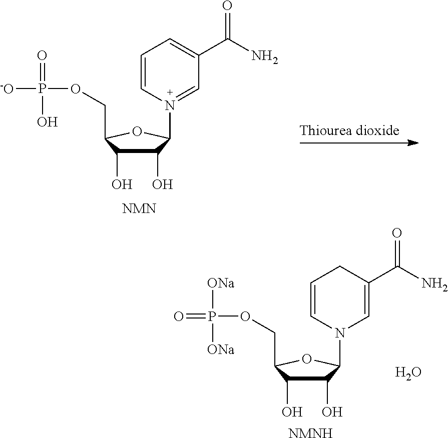

(1) Ammonia water is added to water, followed by the addition of thiourea dioxide and β-nicotinamide mononucleotide (NMN), and the mixture is kept at a reaction temperature;
(2) After the raw material solids disappear, the pH is adjusted to approximately 10.0 with sodium hydroxide solution;
(3) The mixture is concentrated to obtain a first concentrate, which is then purified by column chromatography using sodium chloride solution for elution. The eluate is concentrated to remove salt, yielding a second concentrate;
(4) The second concentrate is cooled to below 10° C., ethanol is added dropwise, and the mixture is stirred to induce crystallization, resulting in crystalline form I of the compound shown in formula I; or the method for preparing the above-mentioned crystalline form I includes the following steps:

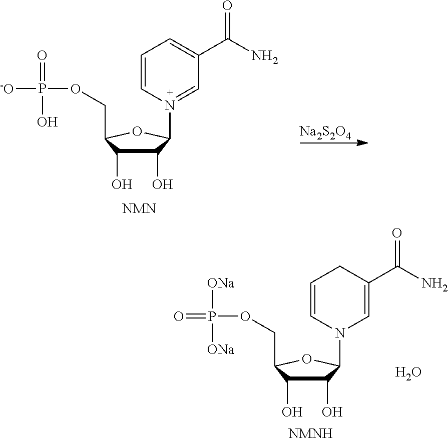

(5) Sodium bicarbonate is dissolved in water, sodium dithionite is added, and then a solution of β-nicotinamide mononucleotide (NMN) in water is added dropwise, and the mixture is kept at a reaction temperature;
(6) After the raw material solids disappear, the pH is adjusted to approximately 10.0 with sodium hydroxide solution;
(7) The mixture is concentrated to obtain a first concentrate, which is then purified by column chromatography using sodium chloride solution for elution. The eluate is concentrated to remove salt, yielding a second concentrate;
(8) The second concentrate is cooled to below 10° C., ethanol is added dropwise, and the mixture is stirred to induce crystallization, resulting in crystalline form I of the compound shown in formula I.

In one embodiment, the column chromatography in step (3) is performed using a resin, preferably an anion exchange resin.

Preferably, step (4) further includes the addition of crystalline form I of NMNH disodium salt hydrate as seed crystals to promote crystallization.

The third aspect of the invention provides a pharmaceutical composition, health food, or cosmetic that includes the above-mentioned crystalline form I as a functional active ingredient.

Further, the above-mentioned pharmaceutical composition, health food, or cosmetic also includes pharmaceutically acceptable excipients or carriers, or excipients or carriers acceptable in health foods or cosmetics.

The above-mentioned pharmaceutical composition, health food, or cosmetic can be used for anti-aging, i.e., for the treatment and/or prevention of age-related degenerative diseases.

Compared with the hydrate crystalline forms A, B, and C with uncertain numbers of water molecules in the prior art CN115368423A, the crystalline form I of the compound of formula I in the present invention has better stability, enhanced hygroscopic resistance, and is more suitable for storage and transportation. It is suitable for large-scale production and helps reduce the production cost of reduced nicotinamide mononucleotide. It can be used to prepare anti-aging pharmaceutical compositions, health foods, or cosmetics.

### BRIEF DESCRIPTION OF DRAWINGS

FIG. 1 is an X-ray powder diffraction pattern (XPRD pattern) of crystal form I of NMNH disodium monohydrate according to the present invention.

FIG. 2 is a differential scanning calorimetry pattern (DSC pattern) of crystal form I of NMNH disodium monohydrate of the present invention. The abscissa is temperature (° C.), and the ordinate is heat flow rate (W/g).

FIG. 3 is a thermogravimetric analysis pattern (TGA pattern) of crystal form I of NMNH disodium salt monohydrate of the present invention.

FIG. 4 is a mass spectrometry pattern (MS) of crystal form I of NMNH disodium salt monohydrate of the present invention.

FIG. 5 is a proton nuclear magnetic resonance spectrum (¹HNMR) of crystal form I of NMNH disodium monohydrate of the present invention.

FIG. 6 is a carbon-13 nuclear magnetic resonance spectrum (¹³CNMR) of crystal form I of NMNH disodium monohydrate of the present invention.

FIG. 7 is a high-performance liquid chromatography pattern (HPLC) of crystal form I of NMNH disodium monohydrate of the present invention.

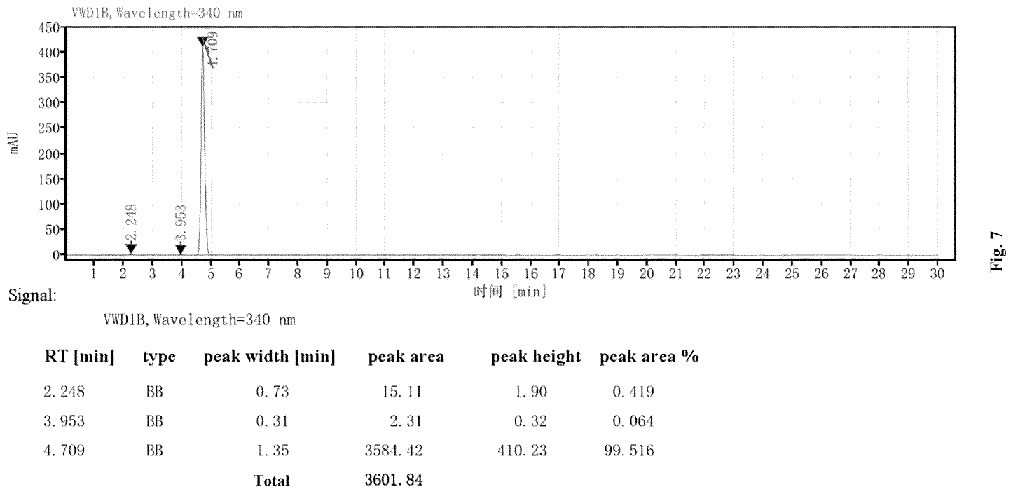

FIG. 8 is an X-ray powder diffraction pattern (XPRD pattern) of crystal form B of NMNH disodium in the comparative example.

FIG. 9 is a differential scanning calorimetry pattern (DSC pattern) of crystal form B of NMNH disodium in the comparative example.

FIG. 10 is a thermogravimetric analysis pattern (TGA pattern) of crystal form B of NMNH disodium in the comparative example.

### DESCRIPTION OF THE EMBODIMENTS

The crystalline form I of the reduced nicotinamide mononucleotide disodium monohydrate is a hydrate. In the pharmaceutical industry, hydrates have unique properties that warrant separate discussion in the synthesis of active pharmaceutical ingredients, drug formulation, storage, and evaluation of drug activity.

The crystalline form I of the reduced nicotinamide mononucleotide sodium salt hydrate has a clear chemical composition, resulting in stable physicochemical properties. The characterization of crystalline form I is primarily indicated by the X-ray powder diffraction pattern (XRPD), which is summarized in Table 1.

The invention further provides a pharmaceutical composition, health food, or cosmetic that includes crystalline form I as an active ingredient. The pharmaceutical composition, health food, or cosmetic may also include pharmaceutically acceptable excipients or carriers.

The term “pharmaceutically acceptable carrier” refers to one or more compatible solid or liquid fillers or gel substances that are suitable for human use and have sufficient purity and low toxicity. “Compatibility” means that the components of the composition can be mixed with the active ingredient of the invention and with each other without significantly reducing the efficacy of the active ingredient. Examples of pharmaceutically acceptable carriers include cellulose and its derivatives (such as sodium carboxymethyl cellulose, sodium ethyl cellulose, cellulose acetate, etc.), gelatin, talc, solid lubricants (such as stearic acid, magnesium stearate), calcium sulfate, vegetable oils (such as soybean oil, sesame oil, peanut oil, olive oil, etc.), polyols (such as propylene glycol, glycerol, mannitol, sorbitol, etc.), emulsifiers (Tween), wetting agents (such as sodium lauryl sulfate), coloring agents, flavoring agents, stabilizers, antioxidants, preservatives, pyrogen-free water, etc.

The dosage forms of the pharmaceutical composition are not particularly restricted and include, but are not limited to, oral and injectable dosage forms (parenteral dosage forms), topical medications such as transdermal absorption dosage forms, etc.

Oral solid dosage forms include capsules, tablets, pills, powders, and granules. In these solid dosage forms, the active ingredient, NMNH sodium salt hydrate crystalline form I, is mixed with at least one conventional inert excipient (or carrier), such as sodium citrate or calcium phosphate, or with the following components: (a) fillers or diluents, such as microcrystalline cellulose, starch, lactose, sucrose, glucose, mannitol, and silica; (b) binders, such as hydroxypropyl methylcellulose, alginates, gelatin, polyvinylpyrrolidone, sucrose, and acacia; (c) humectants, such as glycerol; (d) disintegrants, such as agar, calcium carbonate, potato starch or tapioca starch, alginic acid, certain complex silicates, sodium carbonate, cross-linked polyvinylpyrrolidone, cross-linked sodium carboxymethylcellulose; (e) retardants, such as paraffin; (f) absorption accelerators, such as quaternary ammonium compounds; (g) wetting agents, such as cetyl alcohol and monoolein; (h) adsorbents, such as kaolin; and (i) lubricants, such as talc, calcium stearate, magnesium stearate, solid polyethylene glycol, sodium lauryl sulfate, or mixtures thereof. Capsules, tablets, and pills may also contain buffering agents.

Solid dosage forms such as tablets, sugar-coated pills, capsules, pills, and granules can be prepared with coating and shell materials, such as enteric coatings and other materials known in the art. They may contain opacifiers, and the release of the active ingredient can be delayed to be released in a certain part of the digestive tract. Examples of coating components include polymeric substances and waxy substances. If necessary, the active ingredient NMNH sodium salt hydrate crystalline form I may also form microcapsules with one or more of the above excipients.

Liquid dosage forms for oral administration include pharmaceutically acceptable emulsions, solutions, suspensions, syrups, or tinctures. In addition to the active ingredient, liquid dosage forms may contain conventional inert diluents used in the art, such as water or other solvents, solubilizers, and emulsifiers, such as ethanol, isopropanol, ethyl acetate, propylene glycol, 1,3-butylene glycol, dimethylformamide, and oils, especially cottonseed oil, peanut oil, corn germ oil, olive oil, castor oil, and sesame oil or mixtures thereof.

In addition to these inert diluents, the composition may also contain adjuvants, such as wetting agents, emulsifiers, and suspending agents, sweeteners, flavoring agents, and fragrances.

In addition to the active ingredient NMNH sodium salt hydrate crystalline form I, suspensions may contain suspending agents, such as ethoxylated isooctadecanol, polyoxyethylene sorbitol and anhydrous sorbitol esters, microcrystalline cellulose, aluminum methoxide, and agar or mixtures thereof.

Parenteral injectable compositions may contain sterile aqueous or anhydrous solutions, dispersions, suspensions, or emulsions, and sterile powders for reconstitution into sterile injectable solutions or dispersions. Suitable aqueous and non-aqueous carriers, diluents, solvents, or excipients include water, ethanol, polyols, and appropriate mixtures thereof.

Topical dosage forms include ointments, powders, patches, sprays, and inhalants. The active ingredient is mixed with physiologically acceptable carriers and any preservatives, buffers, or propellants as necessary under sterile conditions.

The following examples further illustrate the invention. It should be understood that these examples are provided for illustrative purposes only and do not limit the scope of the invention. Various modifications or adjustments made by those skilled in the art based on the inventive concept should be included within the scope of the invention.

### EXAMPLES

Reagents: The reactants and catalysts used in the examples of the invention are chemically pure and can be used directly or purified as needed. Organic solvents are of analytical purity and used directly. All reagents are purchased from Aladdin Chemical Reagent Company.

X-ray Powder Diffraction: X-ray powder diffraction analysis was performed on a Thermo Fisher Scientific powder X-ray diffractometer using CuKα radiation. The test power was 45 kV×40 mA, with a scanning speed of 5° /min, step width of 0.02°, and a scanning range of 3˜40°(2θ) for continuous 0˜20 scanning.

Differential Scanning calorimetry (DSC) Characterization: Differential scanning calorimetry was performed on a TA Instruments Q2000/2500 DSC instrument under nitrogen gas protection, with a heating rate of 10° C./min and a temperature range from 25° C. to the set endpoint.

Thermogravimetric Analysis (TGA): Thermogravimetric analysis was performed on a TA Instruments Q5000/5500 TGA instrument under nitrogen gas protection, with a heating rate of 10° C./min and a temperature range from room temperature to the set endpoint.

Nuclear Magnetic Resonance (NMR) Spectrometer Models: Bruker AV-500 and Brucker AV-600.

Mass Spectrometer (LC/MS): Agilent 6120 B, with a DAD detector.

NMNH Sodium Salt Hydrate Content Detection Method (HPLC):

### HPLC Conditions:

Instrument: Agilent 1260 Liquid Chromatograph
Wavelength: 340 nm
Injection Volume: 10 μl
Flow Rate: 1 ml/min
Column Temperature: 25° C.
Run Time: 30 min
Post-Run Time: 10 min
Column: Shim-Pack GIST C18-AQ, 4.6×250 mm, 5 μm (or equivalent column)
Mobile Phase A: 0.02M potassium dihydrogen phosphate, adjusted to pH 9.0 with 4 N potassium hydroxide solution (22.4 g→100 ml)
Mobile Phase B: Acetonitrile
Diluent: Mobile Phase A
Gradient Program:

| Time (min) | Mobile Phase A (%) | Mobile Phase B (%) |
|---|---|---|
| 0 | 100 | 0 |
| 8 | 100 | 0 |
| 20 | 30 | 70 |
| 30 | 30 | 70 |

### Example 1: Preparation of NMNH Sodium Salt Hydrate Crystalline Form I

25% ammonia water (500 g, 7.4 mol, 10.0 eq) was dissolved in 900 mL of water, followed by the addition of thiourea dioxide (285 g, 2.64 mol, 3.8 eq). The reaction system was controlled at around 40° C. Nicotinamide mononucleotide (NMN) (235 g, 0.70 mol, 1.0 eq) was added and the mixture was kept at reaction temperature overnight. After the raw materials disappeared, the pH was adjusted to 10.0 with 2 mol/L sodium hydroxide solution. The mixture was concentrated by nanofiltration to 2000 mL, purified by ion exchange resin column (D202 from Shanghai Huazhen Technology Co., Ltd.), eluted with 3% sodium chloride solution, and the eluate was concentrated by nanofiltration to remove salt. The concentrate was cooled to 5˜10° C., ethanol was added dropwise until the system became turbid, and NMNH sodium salt hydrate crystalline form I was used as seed crystals. The mixture was stirred to induce crystallization, filtered, and the filter cake was vacuum dried to obtain NMNH sodium salt hydrate (234 g) with a molar yield of 88%. The water content was measured by Karl Fischer titration (KF) as 6.2%.

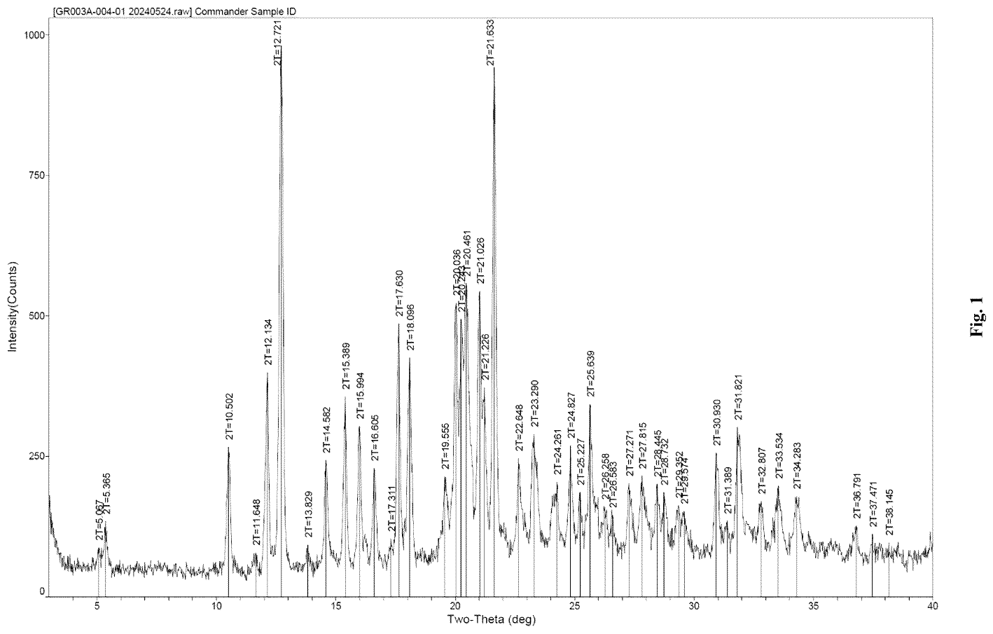

The XRPD pattern of NMNH sodium salt hydrate crystalline form I is shown in FIG. 1, and the absorption peak data are listed in Table 1.

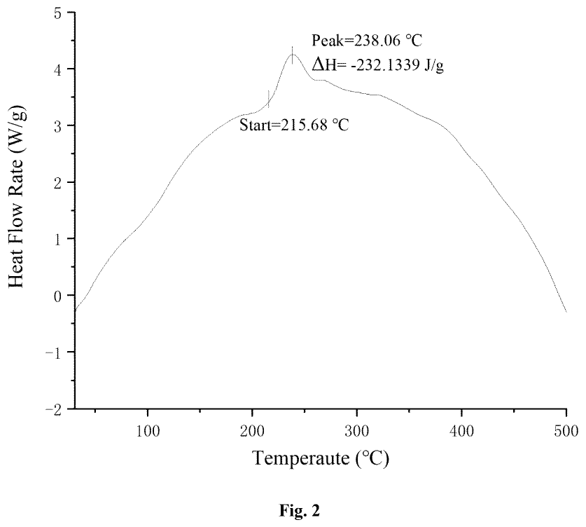

The DSC thermogram of NMNH sodium salt hydrate crystalline form I shows no obvious endothermic peak, but an exothermic peak at 238.06° C., as shown in FIG. 2, indicating the decomposition of NMNH sodium salt hydrate crystalline form I.

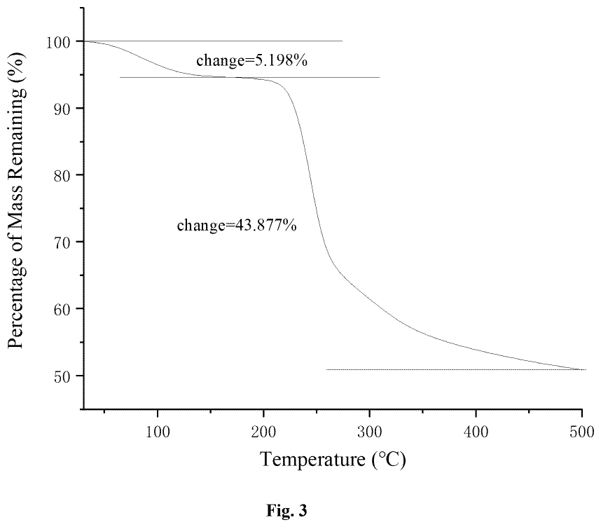

The TGA thermogram of NMNH sodium salt hydrate crystalline form I is shown in FIG. 3. The first step from 50˜160° C. shows a weight loss of 5.198%, corresponding to the loss of crystalline water (the theoretical weight percentage of water in NMNH sodium salt hydrate is 4.523%). The second step from 160˜500° C. shows a weight loss of 43.877%, corresponding to the decomposition of NMNH sodium salt hydrate crystalline form I.

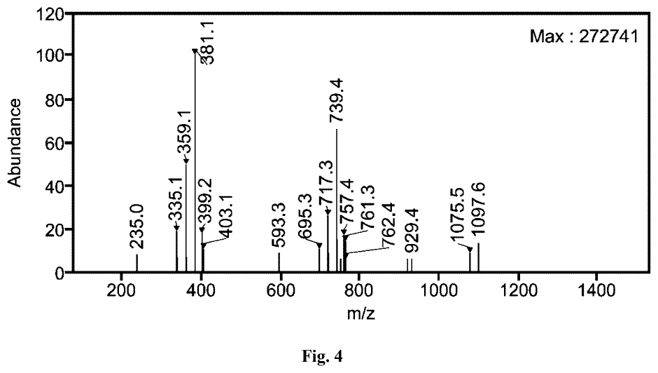

The mass spectrum of NMNH sodium salt hydrate crystalline form I is shown in FIG. 4, showing a main peak at 381, corresponding to the [M+H]⁺ of NMNH sodium salt (theoretical value: 381.04), i.e., C₁₁H₁₆N₂Na₂O₈P⁺; a secondary peak at 359, corresponding to the molecular weight of [M−Na+2H]⁺ with only one sodium ion (theoretical value: 359.05), i.e., C₁₁H₁₇N₂NaO₈P⁺; and a secondary peak at 739, corresponding to the molecular weight of the positive ion complex formed by the above two molecules [2M−Na+2H]⁺ (theoretical value: 739.09), i.e., C₂₂H₃₂N₄Na₃O₁₆P₂⁺.

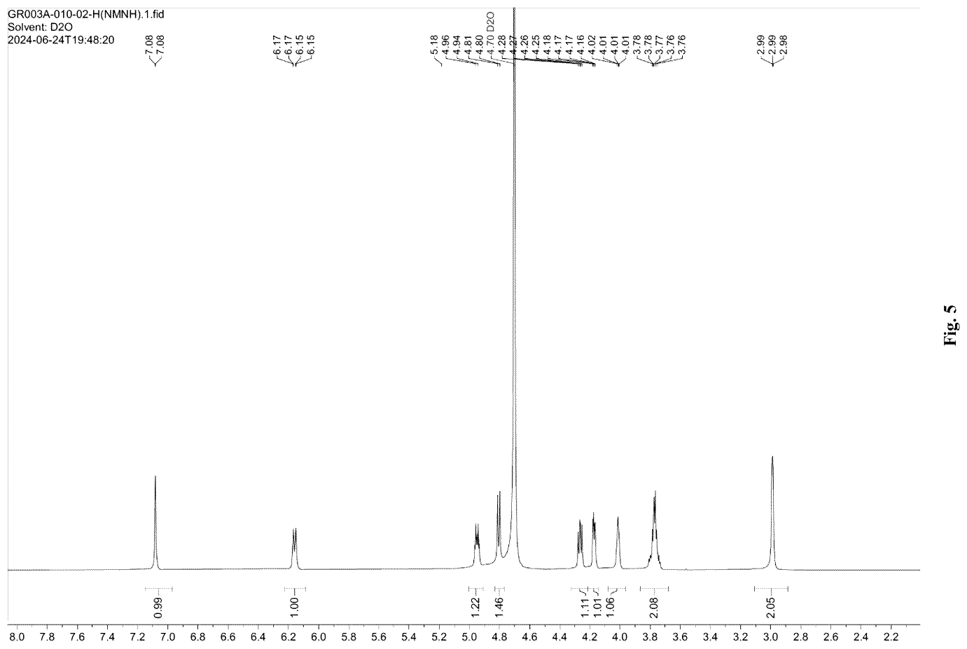

The 1H NMR spectrum of NMNH sodium salt hydrate crystalline form I is as follows: 1H NMR (500 MHz, Deuterium Oxide) δ7.08 (d, J=1.6 Hz, 1H), 6.16 (dd, J=8.2, 1.7 Hz, 1H), 4.95 (d, J=8.2 Hz, 1H), 4.81 (d, J=7.5 Hz, 1H), 4.26 (dd, J=7.5, 5.4 Hz, 1H), 4.17 (dd, J=5.5, 2.1 Hz, 1H), 4.08-3.96 (m, 1H), 3.77 (q, J=4.9, 4.4 Hz, 2H), 3.11-2.89 (m, 2H). As shown in FIG. 5.

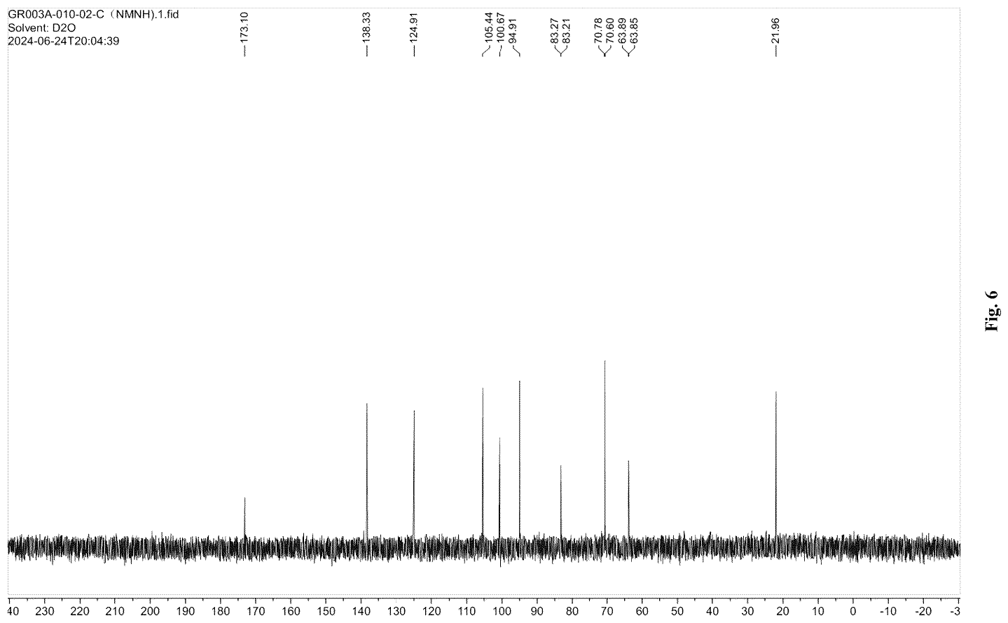

The ¹³C NMR spectrum of NMNH sodium salt hydrate crystalline form I is as follows: 13C NMR (126 MHz, Deuterium Oxide) δ173.10, 138.33, 124.91, 105.44, 100.67, 94.91, 83.27, 83.21, 70.78, 70.60, 63.89, 63.85, 21.96. As shown in FIG. 6.

### Example 2: Preparation of NMNH Sodium Salt Hydrate Crystalline Form I

Sodium bicarbonate (100 g, 1.2 mol, 8.0 eq) was dissolved in 300 mL of water, and sodium dithionite (78.1 g, 0.45 mol, 3.0 eq) was added slowly. The reaction system was controlled at around 40° C. A solution of nicotinamide mononucleotide (NMN) (50.0 g, 0.15 mol, 1.0 eq) in 200 mL of water was added dropwise to the above system and the mixture was kept at reaction temperature overnight. After the raw materials disappeared, the pH was adjusted to 10.0 with 2 mol/L sodium hydroxide solution. The mixture was concentrated by nanofiltration to 500 mL, purified by ion exchange resin column (D202 from Shanghai Huazhen Technology Co., Ltd.), eluted with 3% sodium chloride solution, and the eluate was concentrated by nanofiltration to remove salt. The concentrate was cooled to 5˜10° C., ethanol was added dropwise until the system became turbid, and NMNH sodium salt hydrate crystalline form I was used as seed crystals. The mixture was stirred to induce crystallization, filtered, and the filter cake was vacuum dried to obtain NMNH sodium salt hydrate (46.6 g) with a molar yield of 82%. The spectral data of the prepared NMNH sodium salt hydrate were essentially the same as those of the product in Example 1.

### Example 3: Preparation of NMNH Sodium Salt Hydrate Crystalline Form I Seed Crystals

Nicotinamide mononucleotide disodium salt (10.0 g, 26.3 mmol, 1.0 eq) was added to 16 mL of water and heated to 40° C. to dissolve. Then, anhydrous ethanol (10 mL) was added slowly while stirring and cooling to 5˜10° C. The mixture was kept at this temperature for 24 hours to induce crystallization. The crystals were filtered, washed with cold anhydrous ethanol, and vacuum dried to obtain NMNH sodium salt hydrate crystalline form I (8.9 g) with a molar yield of 85%.

### Comparative Example: Preparation of Crystalline Forms A, B, and C as Described in CN115368423A

Crystalline form A of NMNH sodium salt was prepared according to the method described in Example 1 of CN115368423A. The steps are as follows: Nicotinamide mononucleotide disodium salt (50.0 g, 131 mmol, 1.0 eq) was added to 20 mL of water, and nitrogen gas was blown through the solution at 20˜40° C. After crystals formed, the solid was filtered and dried in a forced-air oven. The resulting solid (44.5g) had a molar yield of 89%. The water content was measured by Karl Fischer titration (KF) as 25.8%.

Crystalline form B of NMNH sodium salt was prepared according to the method described in Example 3 of CN115368423A. The steps are as follows: 30.0 g of the above-obtained crystalline form A of NMNH sodium salt was vacuum dried for 2˜4 hours to obtain crystalline form B (28.8 g). The water content was measured by Karl Fischer titration (KF) as 19.6%.

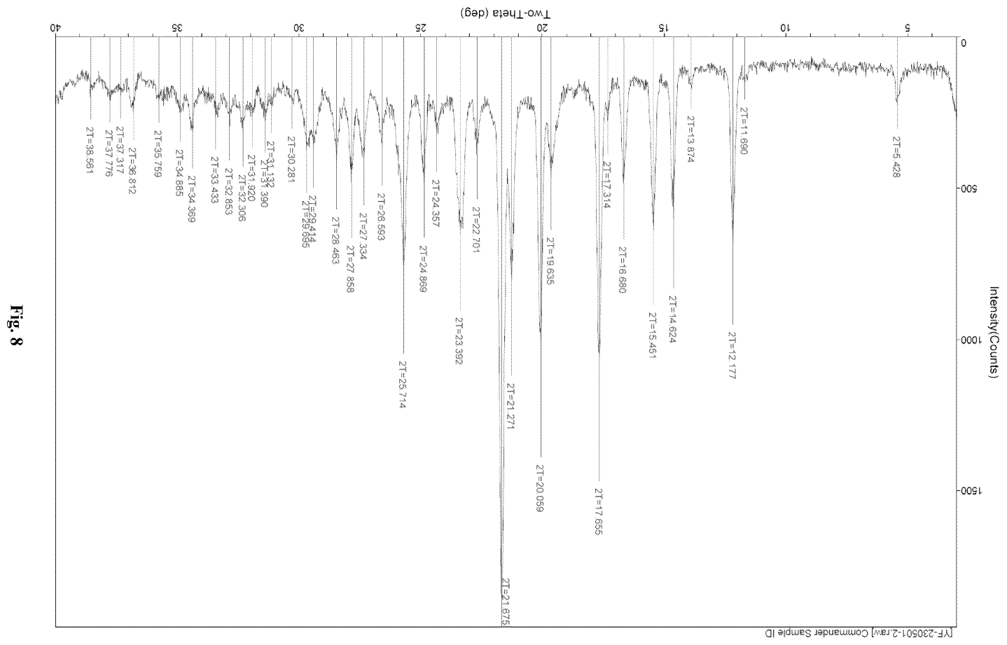

The XRPD pattern of crystalline form B of NMNH sodium salt is shown in FIG. 8.

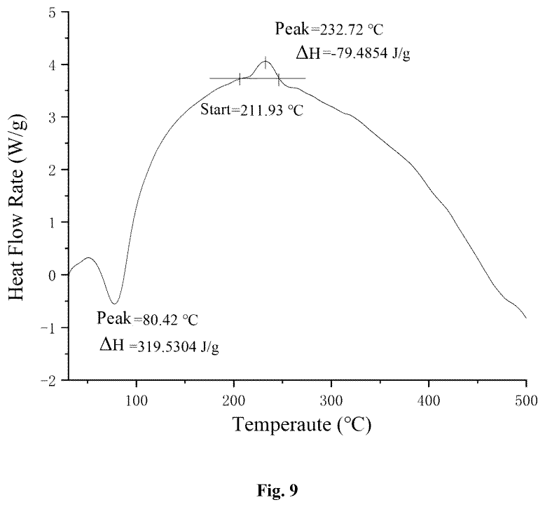

The DSC thermogram of crystalline form B of NMNH sodium salt is shown in FIG. 9.

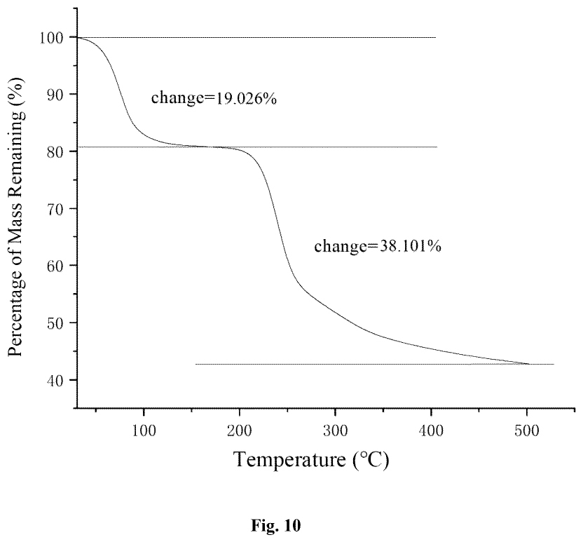

The TGA thermogram of crystalline form B of NMNH sodium salt is shown in FIG. 10.

Crystalline form C of NMNH sodium salt was prepared according to the method described in Example 4 of CN115368423A. The steps are as follows: 15.0 g of the above-obtained crystalline form B of NMNH sodium salt was vacuum dried for 10˜20 hours to obtain crystalline form C (14.4 g). The water content was measured by Karl Fischer titration (KF) as 13.9%.

### Example 4: Thermal Stability Test

The crystalline form I from Examples 1-3 and crystalline forms A, B, and C from the comparative example were placed under conditions of 40° C./75% relative humidity (RH) for 1 week. The purity results measured by HPLC are shown in Table 2:

TABLE 2

| | | HPLC Purity: | HPLC Purity: |
|---|---|---|---|
| Sample | Conditions | Day 0 (%) | 1 Week (%) |
| Crystalline Form I from | 40° C./ | 99.24 | 99.23 |
| Example 1 | 75% RH | | |
| Crystalline Form A from | 40° C./ | 99.32 | 99.21 |
| Comparative Example | 75% RH | | |
| Crystalline Form B from | 40° C./ | 99.30 | 97.22 |
| Comparative Example | 75% RH | | |
| Crystalline Form C from | 40° C./ | 99.23 | 97.11 |
| Comparative Example | 75% RH | | |

The results show that the purity of crystalline form I remained essentially unchanged after 1 week under 40° C./75% RH conditions, and its XRPD pattern remained as shown in FIG. 1, indicating no change in crystalline form. The comparison results in Table 2 demonstrate that crystalline form I has higher thermal stability than crystalline forms A, B, and C.

### Example 5: Hygroscopicity Test

Dynamic moisture adsorption curves were collected on a DVS Intrinsic from Surface Measurement Systems (SMS).

30 mg samples of crystalline form I from Examples 1-3 and crystalline forms A, B, and C from the comparative example were placed in an environment at 30° C./80% relative humidity for dynamic water vapor sorption (DVS) testing. The results measured by HPLC are shown in Table 3:

TABLE 3

| | Water | | Crystal Form |
|---|---|---|---|
| | Adsorption | Hygro- | Change After |
| Sample | (%) | scopicity | DVS Test |
| Crystalline Form I from | 0.20% | Almost non- | No |
| Example 1 | | hygroscopic | |
| Crystalline Form A from | 1.35% | Hygroscopic | Deliquescence |
| Comparative Example | | | |
| Crystalline Form B from | 1.82% | Hygroscopic | Deliquescence |
| Comparative Example | | | |
| Crystalline Form C from | 2.46% | Highly | Deliquescence |
| Comparative Example | | Hygroscopic | |

The above results indicate that the NMNH sodium salt hydrate crystalline form I of the invention has lower hygroscopicity and better stability, making it more convenient for long-term storage and transportation.

The specific embodiments of the invention have been described above. It should be understood that the invention is not limited to the specific embodiments described above. Various modifications or adjustments made by those skilled in the art within the scope of the claims do not affect the essential content of the invention and should be included within the scope of the invention.

1. A crystalline form of the reduced nicotinamide mononucleotide disodium monohydrate as shown in formula I, named crystalline form I, wherein its X-ray powder diffraction spectrum using CuKα radiation shows diffraction peaks at 2θ angles of 10.502°±0.2°, 12.721°±0.2°, 20.036°±0.2°, and 21.633°±0.2°.

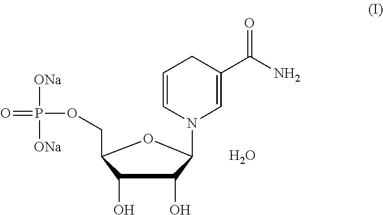

2. The crystalline form according to claim 1, wherein it further has diffraction peaks at 2θ values of 12.134°±0.2°, 15.389°±0.2°, 20.461°±0.2°, and 25.639°±0.2°.

3. The crystalline form according to claim 2, wherein it further has diffraction peaks at 2θ values of 15.994°±0.2°, 17.63°±0.2°, 18.096°±0.2°, 20.243°±0.2°, 23.29°±0.2°, and 31.389°±0.2°; preferably, the XRPD pattern of crystalline form I is substantially as shown in FIG. 1.

4. The crystalline form according to claim 1, wherein in DSC analysis, it has an endothermic peak in the range of 215.68˜251.58° C.; preferably, the maximum absorption peak is at 238.06° C.; more preferably, the DSC pattern of the crystalline form is substantially as shown in FIG. 2.

5. The crystalline form according to claim 1, wherein in TGA analysis, it shows a weight loss of 5.198% in the range of 50˜160° C. and a weight loss of 43.877% in the range of 160˜500° C.; preferably, its TGA pattern is substantially as shown in FIG. 3.

6. A method for preparing the crystalline form as defined in claim 1, wherein it includes the following steps: 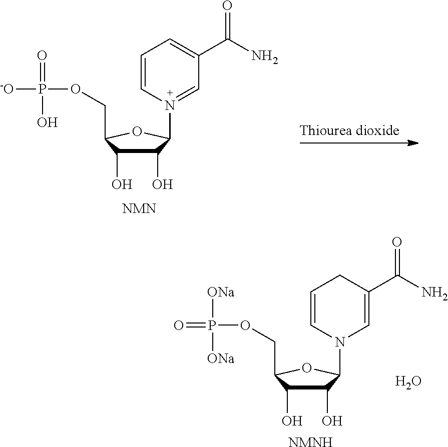
(1) Ammonia water is added to water, followed by the addition of thiourea dioxide and nicotinamide mononucleotide (NMN), and the mixture is kept at a reaction temperature;
(2) After the raw material solids disappear, the pH is adjusted to 10.0 with sodium hydroxide solution;
(3) The mixture is concentrated to obtain a first concentrate, which is then purified by column chromatography using sodium chloride solution for elution. The eluate is concentrated to remove salt, yielding a second concentrate;
(4) The second concentrate is cooled to below 10° C., ethanol is added dropwise, and the mixture is stirred to induce crystallization, resulting in crystalline form I of the compound shown in formula I; or the method includes the following steps: 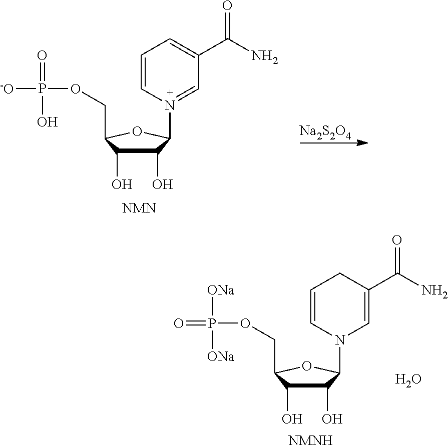
(5) Sodium bicarbonate is dissolved in water, sodium dithionite is added, and then a solution of nicotinamide mononucleotide (NMN) in water is added dropwise, and the mixture is kept at a reaction temperature;
(6) After the raw material solids disappear, the pH is adjusted to 10.0 with sodium hydroxide solution;
(7) The mixture is concentrated to obtain a first concentrate, which is then purified by column chromatography using sodium chloride solution for elution. The eluate is concentrated to remove salt, yielding a second concentrate;
(8) The second concentrate is cooled to below 10° C., ethanol is added dropwise, and the mixture is stirred to induce crystallization, resulting in crystalline form I of the compound shown in formula I.

7. The method according to claim 6, wherein an anion exchange resin is used for the column chromatography in step (3).

8. The method according to claim 6, wherein step (4) further includes the addition of crystalline form I of NMNH sodium salt hydrate as seed crystals.

9. A pharmaceutical composition, health food, or cosmetic, wherein it comprises crystalline form I as defined in claim 1 as an active ingredient.

10. The pharmaceutical composition, health food, or cosmetic according to claim 9, wherein it is used for anti-aging, i.e., for the treatment and/or prevention of age-related degenerative diseases.

11. The pharmaceutical composition, health food, or cosmetic according to claim 9, wherein the crystalline form further has diffraction peaks at 2θ values of 12.134°±0.2°, 15.389°±0.2°, 20.461°±0.2°, and 25.639°±0.2°.

12. The pharmaceutical composition, health food, or cosmetic according to claim 11, wherein the crystalline form further has diffraction peaks at 2θ values of 15.994°±0.2°, 17.63°±0.2°, 18.096°±0.2°, 20.243°±0.2°, 23.29°±0.2°, and 31.389°±0.2°; preferably, the XRPD pattern of crystalline form I is substantially as shown in FIG. 1.

13. The pharmaceutical composition, health food, or cosmetic according to claim 9, wherein in DSC analysis, the crystalline form has an endothermic peak in the range of 215.68˜251.58° C.; preferably, the maximum absorption peak is at 238.06° C.; more preferably, the DSC pattern of the crystalline form is substantially as shown in FIG. 2, wherein it comprises crystalline form I as defined in claim 4 as an active ingredient.

14. The pharmaceutical composition, health food, or cosmetic according to claim 9, wherein in TGA analysis, the crystalline form shows a weight loss of 5.198% in the range of 50˜160° C. and a weight loss of 43.877% in the range of 160˜500° C.: preferably, its TGA pattern is substantially as shown in FIG. 3.

15. The pharmaceutical composition, health food, or cosmetic according to claim 11, wherein it is used for anti-aging, i.e., for the treatment and/or prevention of age-related degenerative diseases.

16. The pharmaceutical composition, health food, or cosmetic according to claim 12, wherein it is used for anti-aging, i.e., for the treatment and/or prevention of age-related degenerative diseases.

17. The pharmaceutical composition, health food, or cosmetic according to claim 13, wherein it is used for anti-aging, i.e., for the treatment and/or prevention of age-related degenerative diseases.

18. The pharmaceutical composition, health food, or cosmetic according to claim 14, wherein it is used for anti-aging, i.e., for the treatment and/or prevention of age-related degenerative diseases.
